(19) United States
(12) Patent Application Publication
(10) Pub. No.: US 2003/0204489 A1
(43) Pub. Date: Oct. 30, 2003

(54) System for the management of information

(75) Inventors: Dominique Vadrot, Fontenay Sous Bois (FR); Martine Verdoux, Paris (FR)

Correspondence Address: YOUNG & THOMPSON, 745 SOUTH 23RD STREET 2ND FLOOR, ARLINGTON, VA, 22202

(73) Assignee: PATIENT ON LINE, Paris (FR)

(21) Appl. No.: 10/424,944

(22) Filed: Apr. 29, 2003

(30) Foreign Application Priority Data
Apr. 30, 2002 (FR) 02 05469

(51) Int. Cl. G06F 7/00; G06F 17/30; G06F 17/60

(52) U.S. Cl. 707/1; 705/3

(57) ABSTRACT

The invention relates to a system for the management of information. and in particular medical information, each item of information concerning a first entity and having been generated by a second entity. It comprises:

means for creating at least one event bringing together in an indissociable manner in one and the same data item:

an item of information concerning the first entity;

an identifier of the first entity; and

an identifier of the second entity; and

means for definitive storage of the content of the or each validated event, each by way of a data item in the or each database.

Access to an item of information is possible solely by an entity for which the identifier is included in the data item containing said item of information.

### TECHNICAL FIELD

The present invention relates to a system for the management of information, and in particular medical information, each item of information concerning a first entity and having been generated by a second entity, the system comprising:

at least one database for the storage of said items of information; and

at least one user station comprising:

means for collecting

at least one item of information;

an identifier of a first entity concerned by the or each item of information; and

an identifier of a second entity who generated said item of information; and

means for access to the or each database for the consultation of said items of information.

### BACKGROUND TO THE INVENTION

In numerous fields it is necessary to be able to ensure confidential storage and authorised and controlled consultation of validated items of information concerning a person.

This items of information may be for example medical information concerning a patient. These items of medical information are generated by one or several medical practitioners subject to the obligations of a professional code of ethics. In particular, these obligations impose upon the practitioners the respect for professional confidentiality, such that practitioners are forbidden to make this information accessible without the authorisation of the patient concerned and the patient must be able to access the information concerning him.

The information management systems which are currently known use relational databases in which there are memorised, on the one hand, all the items of information which must be generated by the system, and, on the other hand, identifiers of the patient concerned and/or the practitioner who has generated these items of information.

In relational databases, the database is organised as a function of the relationships which exist between the data items.

Thus an item of information concerning a patient, the identity of this patient or the identity of the practitioner who generated this item of information is considered as a data item and the links translating the existing relationships between the data items are memorised in the database.

This type of database is convenient since it permits modification of the links existing between the data items, which permits the application of new processing operations to already existing items. This reduces the redundancy of data items contained in the database.

However, the management of the confidentiality necessary for the limitation of the consultation of the information, imposed in particular by the code of ethics of the persons generating the information, is difficult to ensure, due to the multitude of links which can be created in such a database.

The object of the invention is to propose a system of management of information in which the security concerning the confidentiality of the items of information contained in the system is improved whilst permitting easy access to the items of information for authorised persons.

### SUMMARY OF THE INVENTION

To this end, the invention relates to a system of management of information of the aforementioned type, wherein it comprises:

means for creating at least one event bringing together in an indissociable manner in one and the same data item:

the or each item of information concerning a first entity;

an identifier of the first entity; and

an identifier of a second entity;

means for validation of said event by the second entity;

means for definitive storage of the content of the or each validated event, each by way of a data item in the or each database;

and in that said means for access comprise means for permitting access to an item of information included in a data item solely by an entity for which the identifier is included in the data item containing said item of information.

According to particular embodiments, the system for management of information comprises one or several of the following characteristics:

said means for collection are also adapted for the collection of at least one identifier of a supplementary entity authorised to access the or each item of information contained in at least one event; and

means for integrating into the data item corresponding to the event the or each identifier of the or each supplementary entity authorised to access the items of information;

said means for collection are also adapted for the collection of at least one date; and

means for integrating the date into the data item corresponding to the event;

it comprises means for storing, for each event, an identifier for each entity who has integrated into said data item corresponding to the selected event at least one identifier of a supplementary entity authorised to access the items of information;

it comprises means for collecting at least one supplementary item of information;

means for selection of an event to which the or each supplementary item of information is to be added;

means for addition, in said data item corresponding to the selected event, of the or each supplementary item of information; and

means for storing the completed event by way of a data item in said database;

it comprises means for storing, for each event, an identifier for each entity who has added a supplementary item of information in said data item corresponding to the selected event;

it comprises means for storing, for each event, a date on which a supplementary item of information was added in said data item corresponding to the selected event;

said means for definitive storage include means for detection of image data in the or each item of information contained in an event to be stored in the database, and means for conversion of said image data into a predetermined format, if said image data are in a different initial format from the predetermined format, and means for integrating the image data converted into said predetermined format into the data item corresponding to the event;

said means for collection comprise means for automatic identification in the or each item of information of an identifier of the first entity concerned by the or each item of information;

it comprises means for storing, for each event, an identifier of each entity who has accessed the or each item of information contained in this event; and

it comprises means for storing, for each event, a date on which each entity has accessed the or each item of information contained in this event.

The invention also relates to a method of management of information, each item of information concerning a first entity and having been generated by a second entity, the system comprising at least one database for the storage of said items of information and at least one user station comprising means for collecting data and means for accessing the database for the consultation of said items of information, the method comprising:

a step of collecting from a user station:

at least one item of information;

an identifier of a first entity concerned by the or each item of information; and

an identifier of a second entity who has generated said item of information; and

wherein it comprises:

a step of creating at least one event bringing together in an indissociable manner in one and the same data item:

the or each item of information concerning the first entity;

an identifier of the first entity; and

an identifier of the second entity;

a step of validation of said event by the second entity;

a step of definitive storage or the content of each validated event by way of a data item in the or each database;

and in that access to an item of information included in a data item from a user station is only permitted by an entity for which the identifier is included in the data item containing said item of information.

### BRIEF DESCRIPTION OF DRAWINGS

The invention will be better understood by reading the description which follows, given solely by way of example and with reference to the drawings, in which:

FIG. 1 is a schematic view of a system for the management of information according to the invention;

FIG. 2 is a schematic view illustrating the format of a data item used by the system for the management of information shown in FIG. 1;

FIG. 3 is a flow chart of the principal algorithm implemented in the system according to the invention.

### DESCRIPTION OF PREFERRED EMBODIMENT

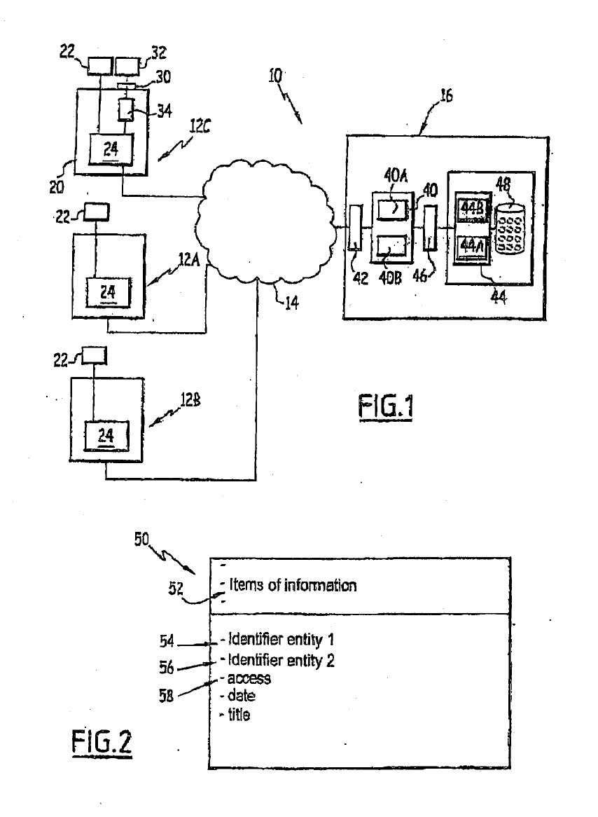

The system for the management of information 10 according to the invention is illustrated schematically in FIG. 1. This comprises, on the one hand, a set of user stations designated by the general reference 12, each connected to a collective information transmission network 14 such as the Internet and, on the other hand, a centre 16 for storage and management of information.

The system for the management of information 10 is intended, in the example under consideration, for the management of medical information concerning identified patients. These items of information are generated by medical practitioners such as doctors, radiologists or biologists in charge of an analysis laboratory.

In particular the management system is adapted to permit the definitive storage of an item of information in the storage centre 16 without it being possible for this item of information to be modified subsequently. Furthermore, at least one identifier of the patient concerned, as well as an identifier of the practitioner who generated the item of information, is preserved, associated with this item of information.

The system permits access to a stored item of information to be given solely to the patient concerned and to the practitioner who generated the item of information, as well as, possibly, after the patient's agreement, to other practitioners.

Each entity entering into the system, whether they be a patient or a practitioner, is equipped with or has access to a user station 12. Thus, for example, a first user station 12A serves the consulting-room of a general medical practitioner and a user station 12B serves the home of a patient. Likewise, for example, a medical imaging laboratory is equipped with a user station 12C.

Each user station 12A, 12B, 12C has a microcomputer 20 equipped with an adapted Internet navigator. It is connected by an adapted interface to the network 14. Each user station includes means 22 for collection of input data such as a keyboard or a data conversion module. From the keyboard it is possible to input in particular an item of medical information, an identifier of a patient such as his name, as well as an identifier of the practitioner who produced the item of information.

Each user station 12 is adapted to implement, from information processing means 24, software means for access to the centre 16 for storage and management of information.

According to the invention, each user station 12 has software means in order to create an event bringing together, in an indissociable manner in one and the same data item, items of information collected concerning a patient, an identifier of the patient and an identifier of the practitioner. These means for creation of an event are advantageously downloaded from the centre 16 and consist for example of a page in HTML (Hyper Text Markup Language) format forming a dialogue interface.

Certain of these user stations, such as the station 12C, have in addition to the microcomputer 20 an interface 30 for connection of the microcomputer to an installation 32 for medical imaging or for collecting medical information capable of producing images or digital items of information in a predefined format such as the format DICOM Hprim HL7. By its nature, this image or digital item of information includes an identifier of the patient concerned. The user station also implements a software module 34 adapted to analyse the digital image produced by the installation 32 and to extract from the latter an identifier of the patient concerned.

The centre for storage and management of information 16 includes a set of servers 40 for the management of access to the centre 16. This set of servers 40 includes in particular an authentication server 40A adapted, as is known per se, to identify the origin of a request addressed to the server centre. It also includes one or several servers 40B adapted to the management of the exchange of executable files and of HTML pages according to the HTTP protocol between the centre for storage and management 16 and the user stations. In particular, the or each server 40B includes a software module adapted to ensure the downloading in each requesting user station of HTML pages constituting user interfaces permitting access to the stored information, as well as the saving of new items of information. This set of servers 40 is connected directly to the network 14 via a first security barrier 42 (firewall).

The set of servers for management of access 40 is also connected to a set of servers 44 for management of events via a second security barrier 46 (firewall). In particular, the set of servers 44 is adapted to implement a software module 44A for transcription of the digital images received in formats different in particular from the DICOM format into one and the same format, for example the XML format.

The set of servers 44 is also adapted to implement a software module 44B for management of the storage of events in a unit 48 for storage and management of access to these events.

This storage unit 48 is intended for the permanent memorisation of one or several databases, the data items of which are constituted by events defined by the user stations and include in particular the items of information to be saved.

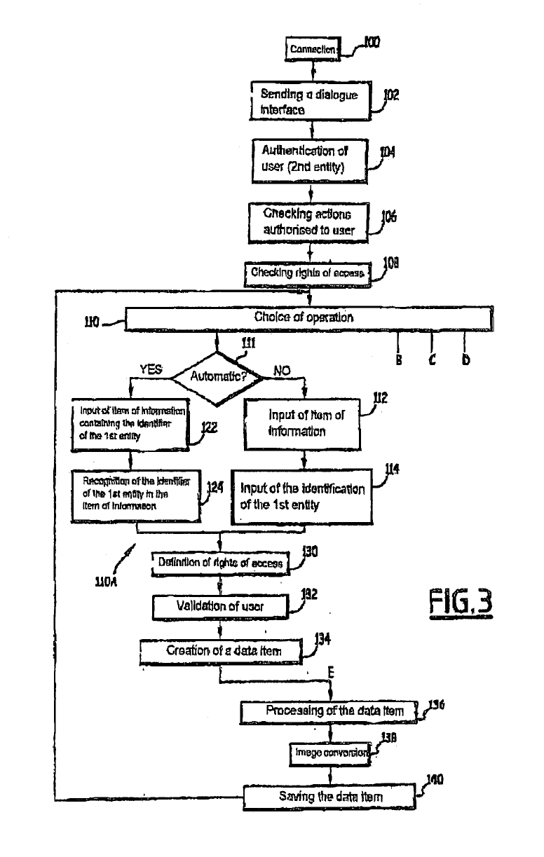

The structure of a data item stored in the database 48 is represented schematically in FIG. 2. This data item corresponds to an event.

Each event comprises at least one actual item of information 52. This item of information is constituted for example by digital data corresponding to the result of an analysis or by a text corresponding to the opinion of a practitioner on the clinical condition of a patient. An item of information may equally be constituted by a file attached to the event such as a document in HTML format or an image file in DIBCOM format or an attachment in an office administration format.

Furthermore, each event includes an identifier 54 of a first entity. This identifier designates the patient concerned by the items of information 52. Likewise, the event includes an identifier 56 of a second entity. This identifier designates the practitioner who produced the item of information.

Each event advantageously includes a list 58 of identifiers of additional entities which can have access to the information.

The event also includes, advantageously but not obligatorily, other items of information to be filled in by the user such as:

a title;

a date of creation and/or of supplements to the event; and

a list of keywords.

For adding an item of information in the storage centre, complementing a pre-existing item of information with a supplementary item of information, modification of the rights of access to an item of information or consulting an item of information, the user connects from a user station 12 to the storage centre 16.

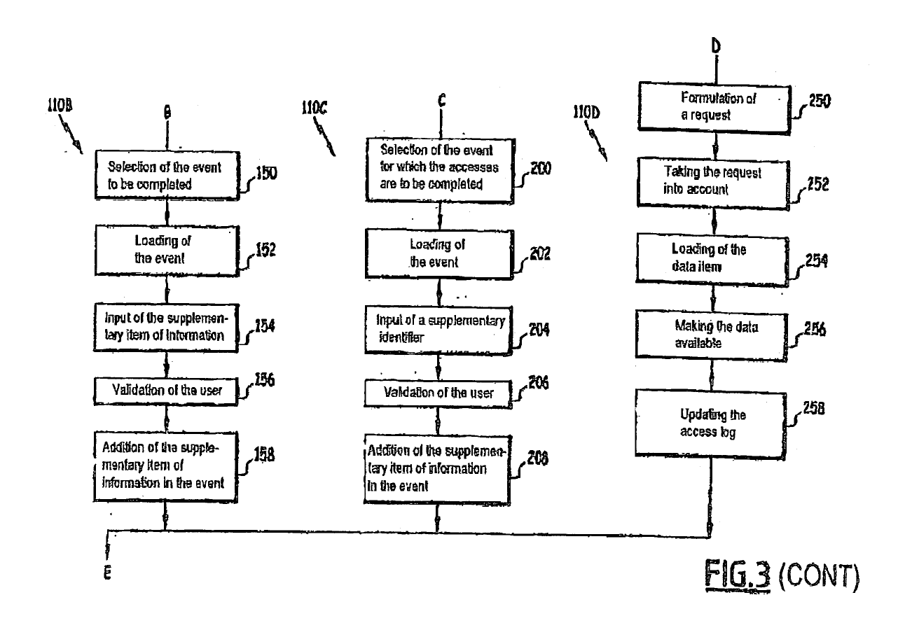

The algorithm of FIG. 3 is then implemented.

The user station can be constituted, for the simplest operations, solely by a microcomputer connected to the Internet with the aid of a navigator of any adapted type. After connection of the user station, at step 100, the set of servers 40 of the storage centre 16 returns a dialogue interface in HTML format to the user station 12, at step 102. At step 104, the centre 16 proceeds via the dialogue interface implemented by the user station with an authentication of the user. As a function of the identifier input by the user, checks of the actions authorised for this user are carried out, at step 106, and a check of the access rights of the user is carried out, at step 108.

The user is then free to proceed with several operations as a function of the actions which are authorised for him. From the interface made available to him he proceeds, at step 110, with the choice of an operation to carry out.

This may be the input of a new item of information into the storage centre 16. The branch 110A of the flow chart is then implemented.

It may equally be the addition of a supplementary item of information in order to complete an item of information already present in the storage centre 16. The branch 110B of the flow chart is then implemented.

The practitioner user can equally modify the rights of access to the items of information stored by authorising a new practitioner to access the information concerning a patient. The branch 110C of the flow chart is then implemented.

Equally, the user can simply gain knowledge of items of information stored in the storage centre by implementation of the branch 110D of the flow chart.

When a practitioner wishes to input a new item of information into the centre 16, the algorithm differs according to whether the item of medical information which the practitioner wishes to input can be associated automatically with a patient constituting the first entity or whether the connection with the patient must be effected manually. This choice is effected at step 111.

If the item of information does not initially contain the identifier of the patient concerned, the item of information is input by the practitioner, for example at the keyboard, at step 112. An identification of the patient concerned is entered, at step 114, particularly by selection of an identifier of the patient from among a list of identifiers of patients or by keystrokes.

By contrast, and in the case of a user station such as the station 12C, the recognition of the identifier of the patient concerned can be achieved automatically at the time of input of the item of information. Thus, the item of information containing the identifier of the patient concerned is input, at step 122, for example via the interface 30. This information is constituted for example by a medical image in DICOM format. At step 124, the software module 36 proceeds to an analysis of the image and a recognition of the identifier of the patient in the image transmitted.

At step 130, the practitioner defines the list of identifiers of the supplementary entities authorised to access the items of information contained in the event. This step consists of defining the list 58 of identifiers of the practitioners authorised to have access.

At step 132, the practitioner validates by entry of a signature code all of the elements constituting the event, namely the actual item of medical information, the identifier of the patient concerned, his own identifier and the list of identifiers of the supplementary entities authorised to have access. At the end of this step, the elements constituting the event can no longer be modified and the event can only be completed.

At step 134, the user station 12 ensures the creation of a data item including the different elements of the event. This data item is encrypted by any suitable process and is addressed by the dialogue interface to the centre 16 for storage and management of information.

Upon its reception, the data item is processed by the servers for management of events 44, at step 136. If the data item contains digital images in formats different from XML format, these images are automatically converted to XML format, at step 138, and the data item is completed by image data in XML format in addition to the image data in another format.

The data item thus reprocessed is saved definitively in the storage unit 48, at step 140.

When the user wishes to complete an event by adding a supplementary item of information, the steps of the branch 110B are implemented after step 110.

At step 150, the event to be completed is selected.

The data item corresponding to the event selected is transmitted by the centre 16 to the user station, at step 152. The data item is only transmitted if the identifier of the user is included in the event in question, whether it be the patient concerned, the practitioner who originated the item of information or a supplementary practitioner whose identifier appears in the list 58.

The supplementary item of information is input at step 154, either manually from the keyboard or by resumption of an already existing file. In this latter case the supplementary item of information constitutes a new attached file.

At step 156, the user validates the addition of information by input of a signature code.

The supplementary item of information is added at step 158 to form a new data element constituting the modified event. Furthermore, the date and the identifier of the user who added the item of information, as well as a link to the item of information are added in the data item in order to follow through the modifications. The new data item thus constituted is then processed in accordance with steps 136 and following.

When the user wishes to modify a right of access, he can solely add new user identifiers authorised to access a given item of information. To this end, the event for which the accesses are to be completed is selected at step 200. The data item corresponding to the selected event is then transmitted to the user station at step 202. The data item is only transmitted if the identifier of the user is included in the event in question, whether it be the patient concerned, the practitioner who originated the item of information or an additional practitioner whose identifier appears in the list 58.

At step 204, the user selects or enters at the keyboard one or several additional identifiers of users authorised to access the item of information then, at step 206, he validates the new identifiers. The supplementary identifiers are added in the data item constituting the event at step 208. Furthermore, the date and the identifier of the user who added the item of information, as well as a link to the new identifiers arc added in the data item in order to follow through the modifications. The steps 136 and following are then implemented again.

For consultation of the items of information stored in the centre 16, and from any user station whatsoever, the steps of the branch 110D are implemented.

At step 250, a request is formulated by the user from the user station. This is taken into account by the servers for management of the events 44, at step 252. As a function of the rights of access contained in the event in question in the request, and as a function of the rights of the user, the content of the data item is transmitted from the storage centre 16 to the user station 12, at step 254.

In particular, the data item is only transmitted if the identifier of the user is included in the event in question in the request, whether it be the patient concerned, the practitioner who originated the item of information or an additional practitioner whose identifier appears in the list 58.

The item of information is then made available to the user at step 256, for example by display, or by saving the content of the data item on the hard disk of the user station.

At step 258 a log of the accesses is updated in the centre 16 in order to record the identifier of the user, the nature of the information made available, the date of access provided by the system and any other useful information.

It will be understood that with such a system for the management of information the reliability of access to the items of information is increased, since the actual item of information is associated, in one and the same data item, with an identifier of the patient concerned, an identifier of the practitioner who generated the item of information and, possibly, the identifiers of other entities likely to access the actual item of information.

Such a system for the management of information can be applied in fields other than the medical field and in particular in the legal field. In this case, the second entity is an attorney or counsel, the first entity being the client of the attorney or counsel.

Equally, this system of management can be applied to the management of complex projects. In this case the first entity is the project itself, whilst the second entities are the different participants in the project.

1. System for the management of information, each item of information concerning a first entity and having been generated by a second entity, the system comprising:
at least one database for the storage of said items of information; and
at least one user station comprising:
means for collecting:
at least one item of information;
an identifier of a first entity concerned by the or each item of information; and
an identifier of a second entity who generated said item of information; and
means for access to the or each database for the consultation of said items of information,
wherein it comprises:
means for creating at least one event bringing together in an indissociable manner in one and the same data item:
the or each item of information concerning the first entity;
the identifier of the first entity; and
the identifier of the second entity;
means for validation of said event by the second entity;
means for definitive storage of the content of the or each validated event, each by way of a data item in the or each database;
and in that said means for access comprise means for permitting access to an item of information included in a data item solely by an entity for which the identifier is included in the data item containing said item of information.

2. System for the management of information as claimed in claim 1, wherein said means for collection are also adapted for the collection of at least one identifier of a supplementary entity authorised to access the or each item of information contained in at least one event; and
means for integrating into the data item corresponding to the event the or each identifier of the or each supplementary entity authorised to access the items of information.

3. System for the management of information as claimed in claim 1, wherein said means for collection are also adapted for the collection of at least one date; and
it comprises means for integrating the date into the data item corresponding to the event.

4. System for the management of information as claimed in claim 2, wherein it comprises means for storing, for each event, an identifier for each entity who has integrated into said data item corresponding to the selected event at least one identifier of a supplementary entity authorised to access the items of information.

5. System for the management of information as claimed in claim 1, wherein it comprises means for collecting at least one supplementary item of information;
means for selection of an event to which the or each supplementary item of information is to be added;
means for addition, in said data item corresponding to the selected event, of the or each supplementary item of information; and
means for storing the completed event by way of a data item in said database.

6. System for the management of information as claimed in claim 5, wherein it comprises means for storing, for each event, an identifier for each entity who has added a supplementary item of information in said data item corresponding to the selected event.

7. System for the management of information as claimed in claim 5, wherein it comprises means for storing, for each event, a date on which a supplementary item of information was added in said data item corresponding to the selected event.

8. System for the management of information as claimed in claim 1, wherein said means for definitive storage include means for detection of image data in the or each item of information contained in an event to be stored in the database, and means for conversion of said image data into a predetermined format, if said image data are in a different initial format from the predetermined format, and means for integrating the image data converted into said predetermined format into the data item corresponding to the event.

9. System for the management of information as claimed claim 1, wherein said means for collection comprise means for automatic identification in the or each item of information of an identifier of the first entity concerned by the or each item of information.

10. System for the management of information as claimed in claim 1, wherein it comprises means for storing, for each event, an identifier of each entity who has accessed the or each item of information contained in this event.

11. System for the management of information as claimed in claim 1, wherein it comprises means for storing, for each event, a date on which each entity has accessed the or each item of information contained in this event.

12. Method of management of information, each item of information concerning a first entity and having been generated by a second entity, the system comprising at least one database for the storage of said items of information and at least one user station comprising means for collecting data and means for accessing the database for the consultation of said items of information, the method comprising:
a step of collecting from a user station:
at least one item of information;
an identifier of a first entity concerned by the or each item of information; and
an identifier of a second entity who has generated said item of information; and
wherein it comprises:
a step of creating at least one event bringing together in an indissociable manner in one and the same data item:
the or each item of information concerning the first entity;
the identifier of the first entity; and
the identifier of the second entity;
a step of validation of said event by the second entity;
a step of definitive storage of the content of each validated event by way of a data item in the or each database;
and in that access to an item of information included in a data item from a user station is only permitted by an entity for which the identifier is included in the data item containing said item of information.
